(19) United States
(12) Patent Application Publication
(10) Pub. No.: US 2010/0289282 A1
(43) Pub. Date: Nov. 18, 2010

(54) System for attaching an implement to a person

(76) Inventors: Ronald Avery, JR., Lake Winnebago, MO (US); Jesse Voysey, Lee's Summit, MO (US)

Correspondence Address: DAVID E HERRON II, PO BOX 12457, Overland Park, KS, 66282, US

(21) Appl. No.: 12/465,915

(22) Filed: May 14, 2009

(51) Int. Cl. A61F 4/00; A44C 9/00; B43L 15/00; B43K 23/00

(52) U.S. Cl. 294/25

(57) ABSTRACT

The disclosure shows a system for retaining an implement adjacent a person's hand. The system includes a band (such as a wrist band or finger ring), and a magnet attached to the band. A metal portion surrounds at least a portion of on the implement, making the implement removably engagable with the system.

### SUMMARY OF THE INVENTION

The invention is a system for retaining an implement adjacent a person's hand. The system includes a band (such as a wrist band or finger ring), and a magnet attached to the band. The implement has a metal portion on its exterior. Thus, the implement is removably engagable with the system by bringing the metal portion into contact with the magnet.

Optionally, the magnet may be connected to the finger ring by magnetic attraction; alternatively, it may be adhered by an adhesive or a solder-type connection. Further, the magnet (preferably in a disk-shaped form) and the band may comprise a unitary, monolithic, one-piece structure.

In a preferred embodiment, the implement is a rod-shaped tool of some sort, such as a writing implement, awl, eraser, a pressure gage or the like. In this embodiment, the metal portion is positioned substantially adjacent the center of mass of the implement. The magnet retains the implement in such a way that it retains the implement but allows it to turn about an axis substantially orthogonal to a longitudinal axis of the implement.

This embodiment allows the wearer a source of amusement, in that the wearer can spin the implement near one's hand, yet have the implement within grasp when it is needed.

### BRIEF DESCRIPTION OF THE DRAWINGS

FIG. 1 shows an exploded and perspective view of the components of the system, according to the principles of the invention.

FIG. 2 shows a perspective view isolating the components of the system in an assembled condition.

FIGS. 3 and 3A are comparative perspective views isolating a preferred embodiment of the band of the system.

FIG. 4 is a perspective view showing the components of a second preferred embodiment of the system, shown in an assembled condition.

FIGS. 5 and 5A are comparative perspective views showing the inventive system, as worn by a user.

### DETAILED DESCRIPTION OF THE PREFERRED EMBODIMENTS

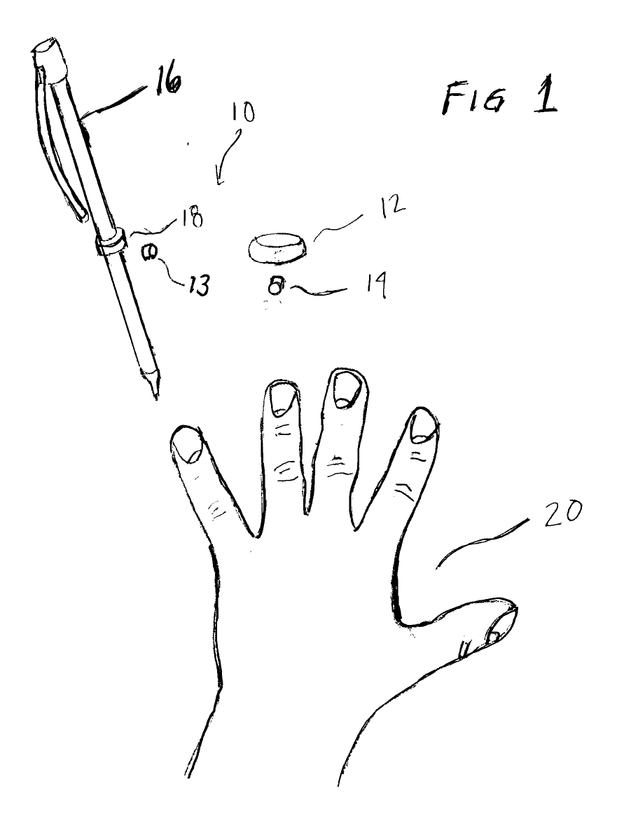

FIG. 1 shows a perspective and exploded view that details the components of the inventive system 10 for keeping an implement 16 adjacent a person's hand 20. The system 10 will include a band 12 and a magnet 14 attached to the band 12. The band 12 may comprise a metal that retains the magnet 14 by a magnetic attraction; alternatively, the magnet 14 may be affixed to a surface of the band 12 by any known method, such as soldering, welding, or adhesive. Alternatively, the band 12 and magnet 14 of the system 10 may comprise a single, unitary, monolithic structure, wherein the entire band is magnetized.

Still referring to FIG. 1, the system 10 will also include an implement 16 having a metallic portion 18 affixed to it. Optionally, the system 10 will also include a magnet 13 that can attach to the metallic portion 18. In preferred embodiments, the implement 16 is a rod-shaped, such as a writing instrument (as shown), or an awl, knife, pick, tire pressure gage, or the like. The implement may have a metallic portion 18 positioned in a generally central location of the implement 16, so that the magnets 13, 14 engage one another and hold the implement 16 in contact with the band 12. Of course, if the system 10 includes optional magnet 13, it is important that the polarities of the magnets 13, 14 be properly configured to attract one another rather than repel one another

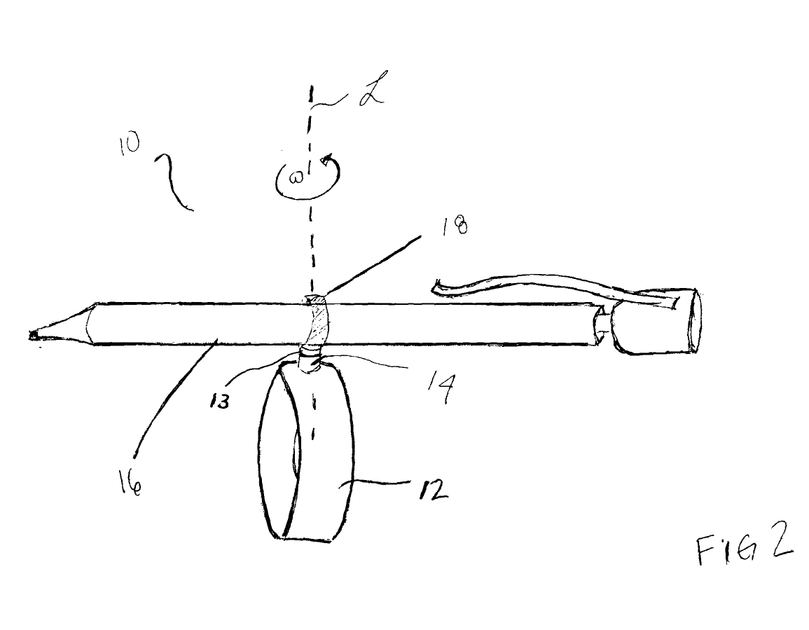

FIG. 2 shows a perspective view of the system 10 shown in an assembled condition. The band 12 is preferably configured to be a finger ring; however, the band may also be worn elsewhere on a wearer's person, such about the wrist. The best mode anticipated for the inventive system, however, is for the band 12 to comprise a finger ring.

Still referring to FIG. 2, the system 10 includes an implement 16 (such as a writing instrument) and a metallic portion 18 positioned generally adjacent the central portion. The metallic portion 18 may be integral with the implement 16, or it could be a sleeve that attaches to the implement 16. In a preferred embodiment, the magnet 13 is adhered to the metallic portion 18 on the implement 16, and the magnet 14 is adhered to the band 12. As another option, the entire implement 16 may be metallic and therefore subject to magnetic attraction. In yet another option, the implement 16 may be entirely made of plastic, and the metallic portion 18 is limited to a magnet 13 embedded within or adhered to the implement.

As shown in FIG. 2, however, the magnets 13, 14 are positioned between the band 12 and the metallic portion 18 of the implement 16; preferably, the magnets 13, 14 are each high-powered, disk-shaped magnets that will retain the implement so that it can rotate with an angular velocity w about axis L, which is generally orthogonal to a longitudinal axis of the implement 16. Other shapes and configurations for the magnets, however, are certainly within the scope of the invention.

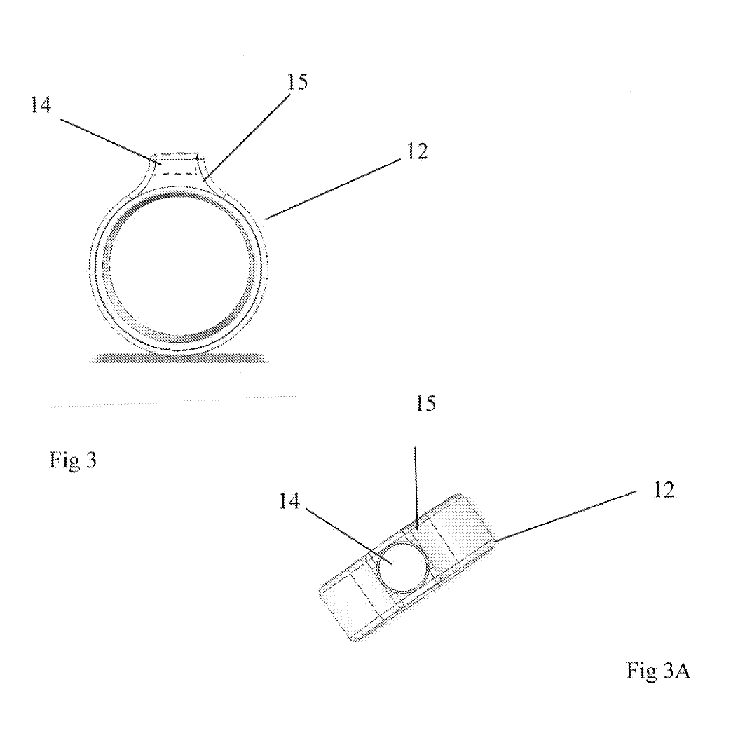

FIGS. 3 and 3A show comparative perspective views of an alternate embodiment of the band 12 portion of the invention. In this embodiment, the band 12 includes a raised portion 15 that houses the magnet 14. In this embodiment, the magnet 14 adheres to the band 12 by fitting tightly within a cooperatively formed vessel in the raised portion 15 of the band 12. Alternatively, the magnet 14 may be retained by the band 12 by any known adhesive.

As evident in FIG. 3, the magnet 14 is embedded within the raised portion 14 such that its top surface may be generally coplanar and flush with the uppermost part of the raised portion 14.

FIG. 3A depicts the band 12, as seen from a vantage point directly above the raised portion 15 of the band. As shown, the magnet 14 is a disk-shaped magnet that is embedded within a cylindrical vessel that is formed in the raised portion 15. Of course, other configurations are within the spirit of the invention.

The embodiments shown in FIGS. 3 and 3A may comprise bands 12 made of any known material, even non-metallic material such as plastic, polymers, or marble.

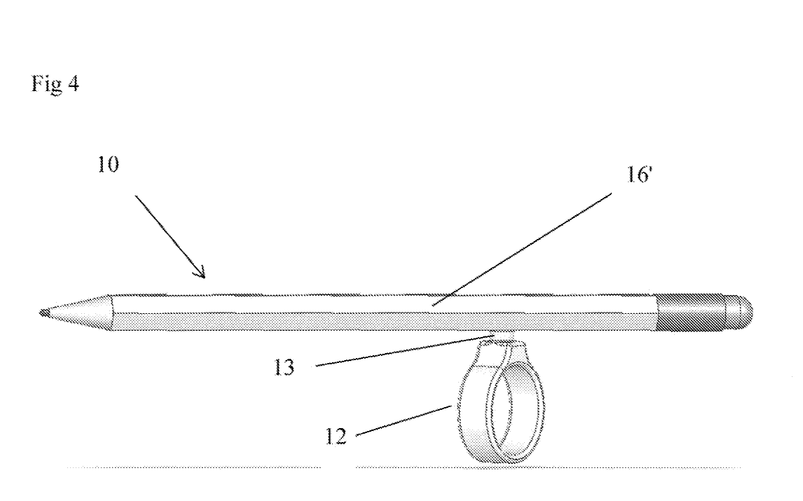

FIG. 4 shows a perspective view of an alternate embodiment of the system 10 in combination with the band 12 depicted in FIGS. 3 and 3A. In this embodiment, a magnet 13 is affixed directly to the implement 16′ without the need for a band 18 or other metallic attachment. In this embodiment, the magnet 13 may be affixed to the implement 16′ by any known method, including an epoxy, an adhesive, or by means of a vessel within the implement 16′ that is cooperatively formed to receive and retain the magnet 13.

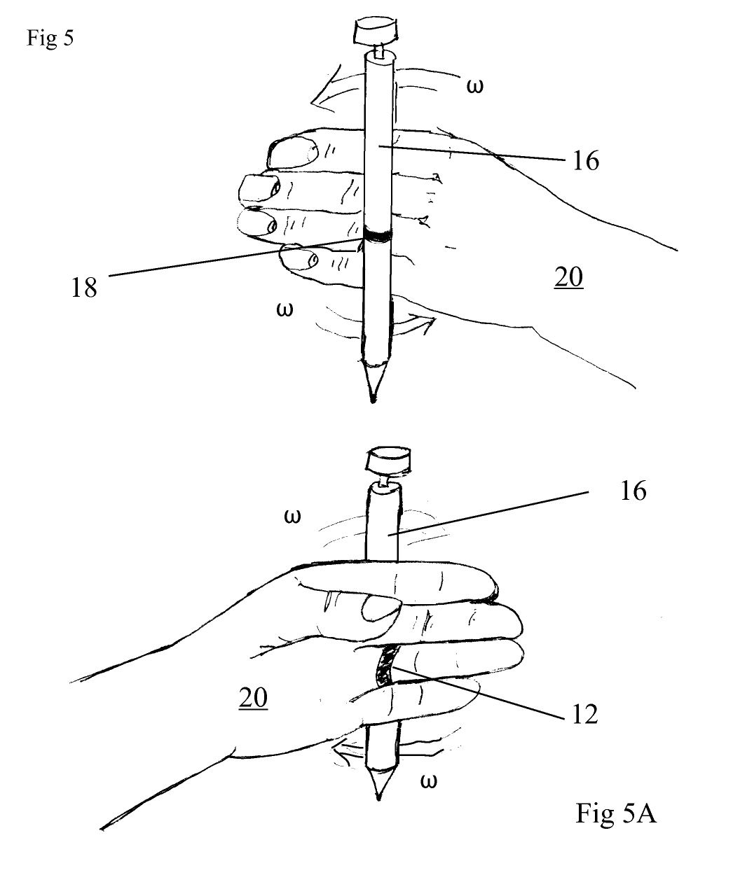

FIGS. 5A and 5B show comparative views of the system 10 in combination with a wearer's hand 20. As shown in FIG. 5A, the implement 16 (a writing instrument) 16 bears a metallic portion 18 positioned at a generally central location on the implement so that it may be free to rotate at an angular speed w about an axis L through the magnet 14 (see FIGS. 2, 4).

Referring to FIG. 5, it is preferred that the magnets 13, 14 have some vertical dimension, which enables the implement to be spaced from the wearer's hand 20, as this space will facilitate free rotation of the implement 16.

Referring specifically to FIG. 5A, the band 12 comprises a finger ring worn on the wearer's hand 20. As aforementioned, however, the band 12 may take a larger form and be worn as a wrist band. When worn on the finger, however, the band 12 enables the implement 16 to turn in direction w about an axis that is generally orthogonal the implement 16 (and the plane of the wearer's hand 20).

Having described the invention in detail, it is to be understood that this description is for illustrative purposes only. The scope and breadth of the invention shall be limited only by patent claims.

1. A system for retaining an implement adjacent a person's hand, the system including
a finger ring;
a first magnet connected to the finger ring;
a metal portion positioned on the implement; wherein, the implement is removably engagable with the finger ring by the engaging the metal portion with the magnet.

2. The system as in claim 1, wherein, the first magnet is connected to the finger ring by magnetic attraction.

3. The system as in claim 1, wherein the magnet is adhered to the finger ring.

4. The system as in claim 1, the first magnet being disk-shaped; and wherein, the metal portion comprises one of a metallic band attached to the implement or a second magnet adhered to the implement.

5. The system as in claim 4, further comprising a second magnet engaging the metallic portion.

6. The system as in claim 1, the implement being rod-shaped; and wherein, the metal portion is positioned substantially adjacent the center of mass of the implement; and wherein, the magnet retains the implement such that it is free to turn about an axis substantially orthogonal to a longitudinal axis of the implement.

7. The system as in claim 6, wherein the implement is a writing implement.

8. A system for retaining a rod-shaped instrument adjacent to a person's hand, the system including:
a band adapted to be worn adjacent the person's hand;
a magnet connected to the band;
a metallic portion positioned on the rod-shaped instrument; wherein,
the instrument is attachable to the band by engaging the metallic portion to the magnet.

9. The system as in claim 8, the band comprising a finger ring.

10. The system as in claim 9, the band comprising a wrist band.

11. The system as in claim 8, wherein the magnet is adhered to the band.

12. The system as in claim 8, wherein the magnet and the band comprise a unitary, monolithic, one-piece structure.

13. The system as in claim 8, further including a metal attachment positioned on the instrument.

15. The system as in claim 8, further including a metallic portion positioned adjacent a center of mass of the instrument; wherein, the magnet engages the metallic portion and enables the instrument to spin about a rotation axis substantially orthogonal a longitudinal axis of the rod-shaped instrument.

16. The system as in claim 8, wherein the instrument is a writing implement.

17. A system, comprising:
a finger ring;
a rod-shaped implement;
a metal sleeve positionable adjacent a center of mass of the implement;
a magnet affixed to the finger ring and configured to engage the metal sleeve;
wherein, the magnet contacts with the sleeve such that the implement is rotatable about an axis through the center of mass and generally orthogonal the rod-shaped implement.

18. The system as in claim 17, wherein the metal sleeve is removably positionable on the implement.

19. The system as in claim 17, wherein the magnet comprises a first magnet part and a second magnet part, wherein, the first magnet part is affixed to the band, and the second magnet part is affixed to the metallic portion.
